(19) United States
(12) Patent Application Publication
(10) Pub. No.: US 2026/0083895 A1
(43) Pub. Date: Mar. 26, 2026

(54) SAFE SURGICAL SUCTION SPONGE

(71) Applicant: Daniel R. Jacobson, Chicago, IL (US)

(72) Inventor: Daniel R. Jacobson, Chicago, IL (US)

(21) Appl. No.: 19/337,531

(22) Filed: Sep. 23, 2025

Related U.S. Application Data
(60) Provisional application No. 63/698,044, filed on Sep. 24, 2024.

(51) Int. Cl. A61M 1/00; A61F 13/20; A61F 13/44; A61M 25/00

(52) U.S. Cl. CPC A61M 1/87; A61F 13/44; A61F 2013/2014; A61M 25/0082; A61M 2205/32

(57) ABSTRACT

Disclosed are devices, systems and methods for a removeable and replaceable gasket and attached patty or sponge for a surgical (or dental) suction cannula which facilitates the safe and effective removal of blood and other bodily fluids from a body cavity while allowing manipulation and/or management of the sponge and/or various tissues therein. These devices protect delicate anatomic structures from excessive negative pressure while allowing effective suctioning of fluid and debris from the operative field.

### CROSS-REFERENCE TO RELATED APPLICATIONS

This application claims the benefit of U.S. Provisional Application No. 63/698,044 entitled “SURGICAL SUCTION DEVICES” filed Sep. 24, 2024, the disclosure of which is incorporated by reference herein in its entirety.

### FIELD OF THE INVENTION

The present subject matter relates generally to surgical suction devices, and more particularly to a removeable and replaceable gasket and attached sponge for a suction device which facilitates the safe and effective removal of blood and other bodily fluids from a body cavity while allowing protection, manipulation, and/or management of various tissues therein.

### BACKGROUND OF THE INVENTION

During surgical operations, it is often desirable to remove fluid such as blood and other body fluids, and debris such as bone dust from the body cavity being operated on. This is commonly done with a suction cannula (also called a suction tip, tube or catheter) connected to a suction source. However, since undesired trauma might result if tissues are drawn into the suction tube, it is customary to lay down a sponge in the body cavity and press the end of the suction tube against the sponge, so that any fluid is drawn into the suction tube through the sponge without picking up or damaging tissue, preventing clogging of the suction tube and preventing contact between delicate tissues and the end of the suction tube. Such a procedure is often time-consuming and inconvenient. The invention allows the surgeon or assistant to place the sponge in position without having to use a second instrument and hand motion. This advantage could be especially useful for endoscopic and robotic procedures.

Moreover, there is often a need for a surgeon to move, shift, separate, dissect, and/or otherwise manipulate anatomical tissues during a surgical procedure. In many cases, manipulation of these tissues can cause and/or induce the release of blood and/or other body fluids, into the surgical cavity, often obscuring the surgeon's view of the surgical field, or worse, damage delicate anatomic structures. It would be desirable to have a surgical instrument capable of safely manipulating anatomical tissues while concurrently removing fluids during a surgical procedure.

Accordingly, there is need for further improvement in surgical instruments and tools, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Because the direct application of strong negative pressure can damage delicate anatomic structures such as nerves, brain tissue, dura, veins, arteries, eyes, cysts, and tumors, especially if such tissues are drawn into a suction tube, the present invention desirably isolates those tissues via a permeable sponge from the suction tube. By adding a protective sponge material at the distal end of the suction tube, the tissue is protected from too much negative pressure.

Various embodiments herein include removeable and/or replaceable devices that attach to a suction device which allow the safe and effective removal of blood and other bodily fluids from a body cavity. Surgical suction is used to clear the operative field of fluids and debris such as blood, irrigation fluid, bone dust, lymph fluid, pus, cerebrospinal fluid, and soft tissue and others. Strong negative pressure can damage delicate anatomic structures such as nerves, brain and small blood vessels, potentially leading to severe surgical complications. Suction as high as −600 mmHg is typical in the operating room.

In one exemplary embodiment, a flexible silicon or similar material gasket is provided having a lumen extending therethrough, with a portion of the gasket attached or otherwise connection to a neurosurgical cottonoid sponge, also called a cottonoid or neuro pattie or similar device, wherein the sponge and/or gasket concurrently provide for the removal of blood and other bodily fluids through the sponge/gasket lumen. The gasket can be attached to a generally rigid suction tube of a standard surgical suction device, such as a Frazier or Fukushima tip, with the gasket and attached sponge being capable of rapid and convenient removal and/or replacement. If desired, gaskets of various diameters can be provided to accommodate different types and sizes of suction cannulas,

In various embodiments, the surgical sponge material and attached gasket can be removable from the suction tip and held by compression, bayonet type and/or other fitting. Ideally, the sponge is constructed of materials already approved for surgical use (Codman, American Surgical, Medline, Weck-cel) such as spun rayon, cotton, cellulose and more. The material will desirably allow clearance of fluids by suction with the tip vacuum pressure decreased at the fluid-tissue interface. If desired, a single sponge could include multiple gaskets at different positions/locations on the sponge.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIGS. 1A through 1F depict various views of one exemplary embodiment of a gasket constructed in accordance with the teachings of the present invention;

FIGS. 2A and 2B depict exploded and assembled perspective views of a gasket and patty, attached to the distal end of a suction tip;

FIGS. 3A through 3F depict various views of another exemplary embodiment of a gasket constructed in accordance with the teachings of the present invention; and

FIGS. 4A through 4F depict various views of another exemplary embodiment of a gasket constructed in accordance with the teachings of the present invention.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,” unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may comprise various surgical materials, including silicone, rubber, latex, or any other biocompatible material, and/or any combination(s) thereof.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it should be readily apparent that more than one component, device and/or article may be used in place of a single component, device and/or article, unless otherwise noted. Similarly, where more than one component, device and/or article is described herein, it should be readily apparent that a single component, device and/or article may be used in place of the more than one component, device and/or article, unless otherwise noted. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

While performing certain surgical procedures, especially in the brain and spine, a surgeon often places a neurosurgical cottonoid sponge, also called a cottonoid or neuro patty, or pattie, over or against the underlying tissue to protect the tissue, absorb body fluids, and irrigation fluids. The surgeon frequently uses a suction aspirator device, often called a suction cannula, or suction tip, or suction catheter, or suction tube, against the surface of the cottonoid sponge for purposes of protection of tissue, and for use as a dissecting tool. Suctioning against such barrier is a common practice in many surgical specialties, especially neurosurgery and spine surgery, but also plastic surgery, and head and neck surgery, microvascular surgery, and hand surgery. As the surgeon proceeds through the surgical field, the protective sponge will often need to be moved, repositioned, or replaced.

Disclosed is a connector or gasket which desirably connects a sponge to the suction tip or cannula of a suction device, via friction fit or similar mechanism, with the gasket linked, connected and/or embedded into the surface of the cottonoid sponge. This arrangement allows the sponge to be moved in concert with the suction tip. The arrangement also desirably minimizes the opportunity for the suction to slip off the sponge in an unwanted manner, thereby increasing the safety of the operation. Various embodiments of the gasket can be designed and/or configured to channel fluids from the surgical field through various points to the suction apparatus. The gasket can be available in various sizes and/or shapes to fit suction cannulas commonly used in the art. The gasket can be adhered to the sponge by any number of mechanisms, including but not limited to adhesives, stitching, stapling and/or the like. The gasket material may comprise a plastic, or silicone, or elastomers, or other materials known to those of skill in the art (including material that may be developed in the future).

Various gasket designs and/or configurations can be provided to desirably inhibit and/or prevent tissue damage while allowing for desired suction. The disclosed gasket and attached cottonoid sponge (and/or other perforated and/or absorbent material) are desirably sized and configured to fit to the distal end of a suction tube tip. In various alternative embodiments, one or more channels or grooves can be cut and/or incorporated into the gasket to allow ingress/egress of fluids.

One significant improvement with the disclosed device is the ability for a surgeon to use the device using a single hand. Generally, the surgeon or assistant will use two hands to manipulate a suction cannula and sponge-one hand on the suction cannula and the other hand, using a forceps or other instrument, to move the sponge. By attaching the suction tip to the gasket and attached sponge, the surgeon only need to move the suction device to reposition the sponge.

Desirably, the gasket and sponge can easily be removed and replaced on the suction tip, allowing the surgeon or technician to quickly and easily exchange a saturated, dirty and/or used sponge with a new gasket/sponge combination during the surgical procedure. Currently, cottonoid patty sponges use standard sizes and commonly used materials, such as cotton and spun rayon, which can be easily incorporated into the present invention. Similarly, the disclosed gaskets could be included in a kit containing standard size gaskets (and/or a plurality of sizes) and could have various diameters through accommodate different types and sizes of suction cannulas. In one exemplary embodiment, the gasket can comprise a plastic, silicone and/or elastomer material for strength and flexibility for secure fit.

The gasket and/or the sponge will desirably incorporate a radio-opaque marker for identification in a surgical field. Surgical sponges are typically embedded with some technology to identify the sponge's presence within surgical wounds or body cavities. Generally, sponges are embedded with a radio-opaque marker. In various embodiments, the disclosed devices would also incorporate similar identifying features to desirably decrease the chance of retention. Accordingly, the gasket should be securely attached to the sponge to avoid separating from the sponge and getting lost in the patient. If desired, the gasket itself could also be made with a radiopaque marker embedded, to make it identifiable under radiographic examination.

In various embodiments, the sponge may be similar to a Neuro cottonoid sponge (also called a neuro pattie, pattie and/or patty), which is improved by placing a fenestration with a gasket, grommet or eyelet within the top layer of a laminated sponge to allow egress of fluid from the surgical field without exposing the tissues to excessive vacuum force. The fenestration component is gasketed or compression fitted with a biocompatible material to fit a standard size suction tip, such that any commercially available suction tip could be used with this device.

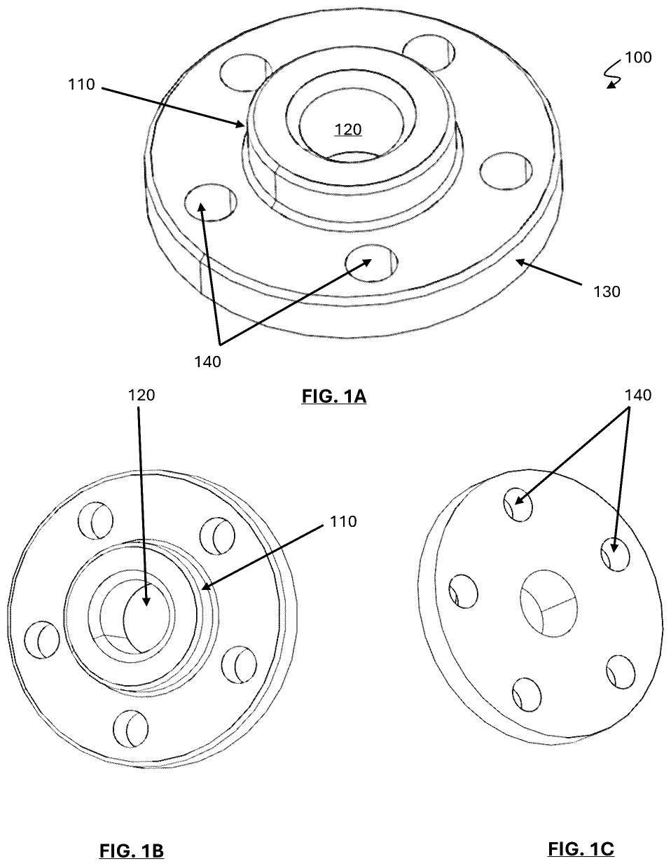

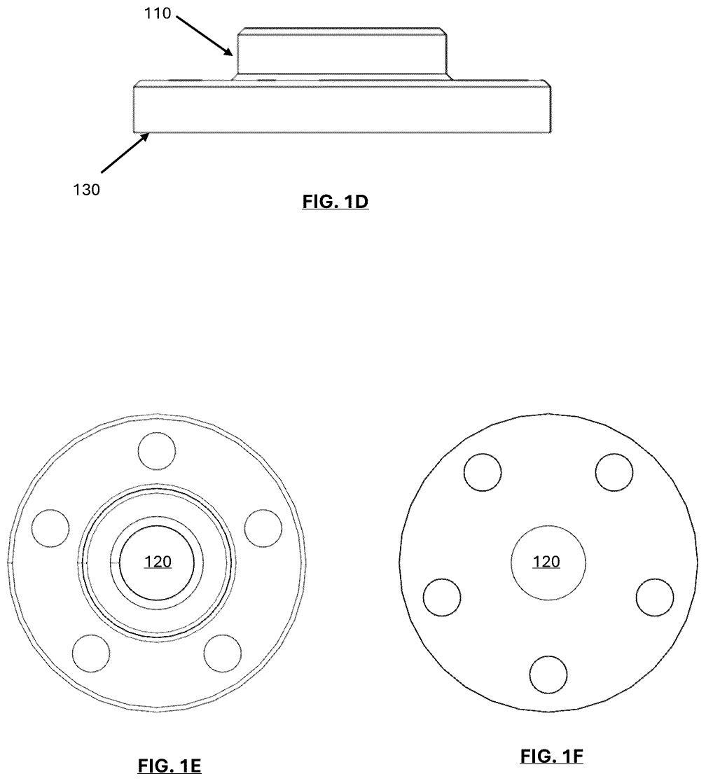

FIGS. 1A through 1F depict views of one exemplary embodiment of a gasket 100 constructed in accordance with teachings of the present invention. The gasket comprises a central body 110 having a lumen 120 extending therethrough, a lower flange body 130, and one or more openings 140 extending through the flange body 130.

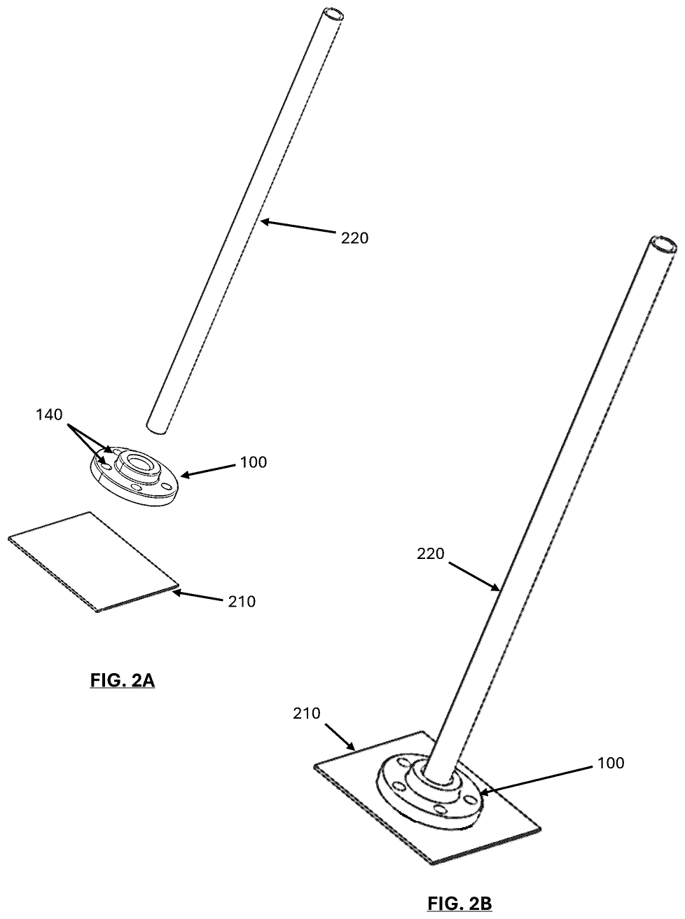

As best seen in FIGS. 2A and 2B, the gasket 100 can be attached to a cottonoid patty 210 or surgical sponge. Desirably, the gasket 100 can be stitched or secured to the patty 210 through the one or more openings 140 extending through the flange body 130. Alternatively, surgical rivets or other fasteners can be utilized. In still other embodiments, the gasket can be bonded to the patty 210 using adhesives or other attachment means know to those of skill in the art.

In use, the gasket 100 and attached cottonoid patty 210 can be pushed onto or otherwise attached to the distal end of a suction tube 220. In the disclosed embodiment, the lumen 120 of the gasket 100 is desirably slightly smaller in inner diameter than the outer diameter of the suction tube 220, such that the lumen will elastically expand around the suction tube and create a compressive/friction fit around the tube. This attachment will desirably allow the gasket 100 and attached cottonoid patty 210 to be moved around the surgical field simply by moving the attached suction tube. When the cottonoid patty 210 has been saturated and/or the suction is clogged by debris, the gasket 100 and attached cottonoid patty 210 can simply be pulled off of the suction tube using moderate force and replaced on the suction tube with another gasket 100 and attached cottonoid patty 210, if desired.

While the gasket is shown as engaged with the suction tube using and expandable compression and/or friction fitting arrangement, it should be understood that other engagement and retention arrangements, such as a barbed adapter, a bayonet fitting, a clip-in mechanism, a screw fitting and/or a luer fitting, as well as other adapter configurations known in the art, could be alternatively utilized. In other embodiments, the suction tube may also incorporate a ridge, tab or indent (not shown) to prevent loosening of the suction tube from the gasket, with the gasket optionally including a mating feature to accommodate the ridge, tab or indent.

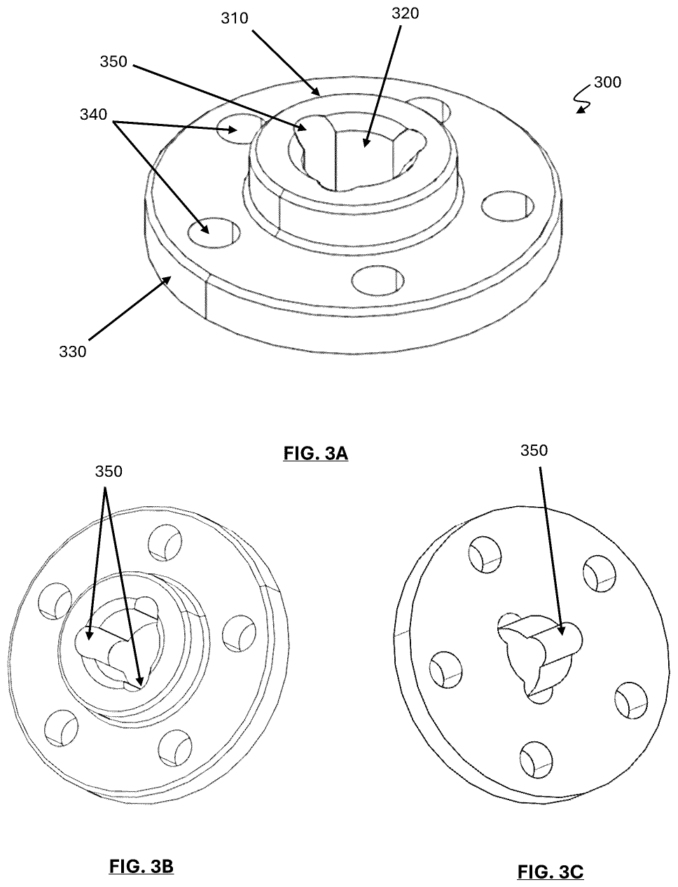

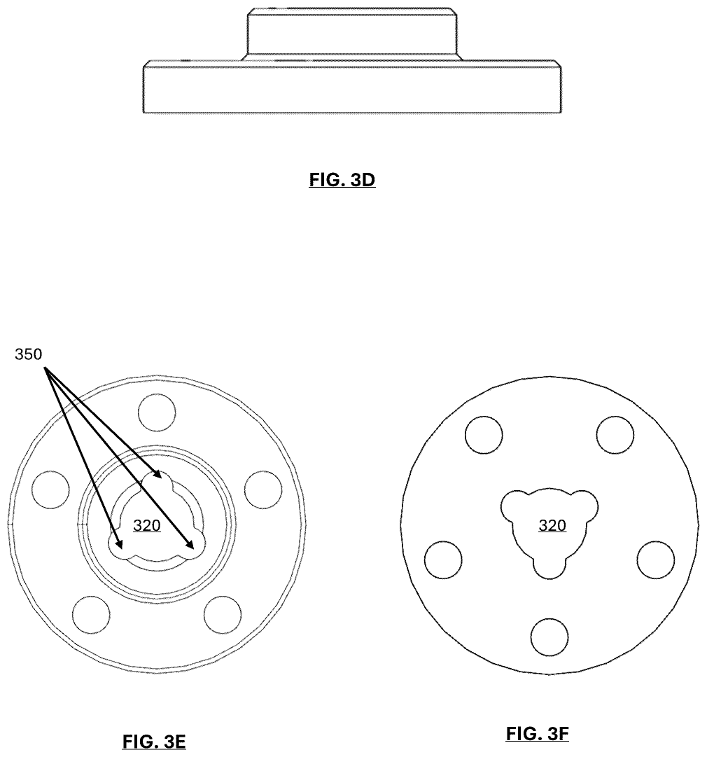

FIGS. 3A through 3F depict views of another exemplary embodiment of a gasket 300 constructed in accordance with teachings of the present invention. The gasket comprises a central body 310 having a lumen 320 extending therethrough, a lower flange body 330, and one or more openings 340 extending through the flange body 330.

In this embodiment, the lumen 320 of the gasket 300 includes one or more channels 350, which channels desirably remain open when a suction tube is placed within the lumen 320. In this embodiment, some of the suction force from the suction tube can redirect through these channels, thereby slightly reducing the localized pressure of the suction force applied through the attached patty, and also allowing the surgeon to suction fluid that need not pass through the patty and into the suction tube.

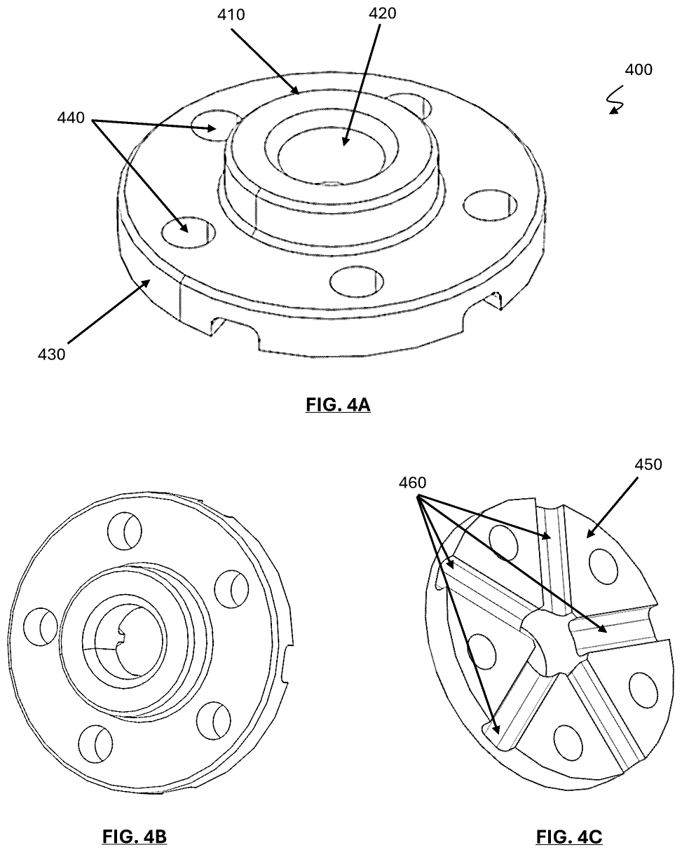

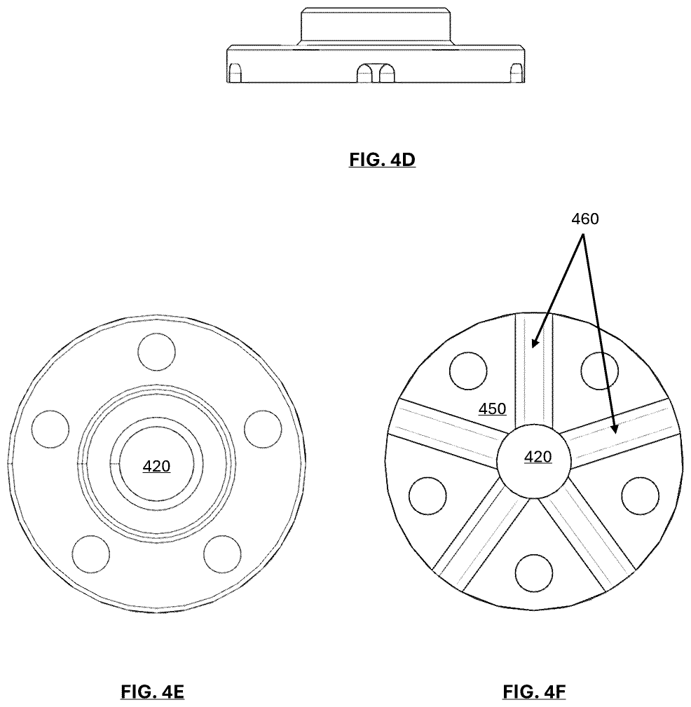

FIGS. 4A through 4F depict views of another exemplary embodiment of a gasket 400 constructed in accordance with teachings of the present invention. The gasket comprises a central body 410 having a lumen 420 extending therethrough, a lower flange body 430, and one or more openings 440 extending through the flange body 430.

As best seen in FIGS. 4C and 4F, in this embodiment a face 450 of the flange body 430 includes one or more channels 460 formed therein, with the channels desirably extending along the face of the patty to which the gasket is attached. Desirably, some of the suction force from the lumen 420 can redirect through these channels 460, thereby increasing the effective surface area of the suction force acting through the patty. In addition, these channels can slightly reduce the localized pressure of the suction force applied through the attached patty, and also allowing the surgeon to suction fluid that need not pass through the patty and into the suction tube.

In the various embodiments disclosed herein, one or more sutures, pins, rivets, grommets, fasteners, and/or other securement devices can extend through the patty and into/through the openings 140 of the gasket. Desirably, these securement devices will retain the gasket on the patty, alleviating any concern for the patty and/or gasket to be left within the surgical field upon completion of the surgical procedure. In still other embodiments, the gasket can be bonded to the patty using adhesives or other attachment means known to those of skill in the art.

Surgical patties and/or sponges are often embedded with some technology to find within surgical wounds or body cavities. Generally, patties are embedded with a radio-opaque marker. If desired, the gasket and/or patty and/or components thereof can incorporate one or markers or other radiopaque elements which can be visualized and/or detected using non-invasive visualization methods. Such markers could be helpful in ensuring that the patty and/or gasket are not inadvertently left within the surgical field upon completion of the surgical procedure. Desirably, the gasket and patty will be securely fashioned to avoid separating from the patty from the gasket and getting lost in the patient. The gasket itself could also be made with a radiopaque marker embedded, to make it identifiable under radiographic examination.

It should be understood that the gaskets and patties described herein can be made and/or assembled in a variety of sizes, shapes and/or configurations to accommodate currently used suction tips and suction capacity. If desired, a plurality of preassembled gaskets and attached patties can be provided in a kit form for use during a surgical procedure.

The present invention provides various components, devices, systems and methods for treating various anatomical structures of the human and/or animal anatomy.

In accordance with another aspect of the present subject matter, various methods for manufacturing and/or assembling a gasket and/or attached patty as set for within any of the details described with the present application are provided.

If desired, a gasket constructed in accordance with the various teaching herein could be constructed from a variety of modular components, including modular components comprising different materials. If desired, such modular components could be provided in a kit form for selection and/or assembly in a surgical theatre and/or in situ during a surgical procedure. If desired, various components described herein may be removable and replaceable.

While embodiments and applications of the present subject matter have been shown and described, it would be apparent that other embodiments, applications and aspects are possible and are thus contemplated and are within the scope of this application. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A surgical tool for use in a body cavity comprising:
a gasket having a proximal end, a distal end and a lumen extending through the gasket from the proximal end to the distal end,
a lower flange positioned at the distal end of the gasket, the lower flange having a plurality of openings extending therethrough,
a permeable patty or sponge attached to the lower flange, at least a portion of the patty or sponge extending across the lumen,
a proximal end of the lumen being dimensioned to receive a suction tube tip of a surgical suction tool therein,
wherein, when a suction force is applied by the surgical suction tube, the suction force draws a fluid from the body cavity through the permeable patty or sponge and into the suction tube.

2. The surgical tool of claim 1, wherein the gasket comprises a flexible material.

3. The surgical tool of claim 2, wherein the lumen is sized smaller in diameter than an outer diameter of the suction tube tip, wherein the flexible material surrounding the lumen elastically expands to accommodate the suction tube tip.

4. The surgical tool of claim 3, wherein the body is retained on the suction tube tip by frictional engagement between an inner wall of the lumen and an outer wall of the suction tube tip.

5. The surgical tool of claim 1, wherein at least one of the gasket and the permeable patty or sponge is impregnated with a radio-opaque marker.

6. The surgical tool of claim 1, wherein at least a portion of the permeable patty or sponge is attached to a distal face of the lower flange.

7. The surgical tool of claim 6, wherein the portion of the permeable patty or sponge attached to the distal face of the lower flange is secured to the lower flange using one or more sutures, stitches or staples extending through the plurality of openings extending through the lower flange.

8. The surgical tool of claim 1, wherein at least a portion of the permeable patty or sponge extends laterally from the gasket.

9. The surgical tool of claim 1, wherein the gasket further comprises one or more channels formed into an inner wall of the lumen.

10. The surgical tool of claim 1, wherein the one or more channels formed into an inner wall of the lumen are oriented parallel to a longitudinal the axis of the lumen.

11. The surgical tool of claim 1, wherein the gasket further comprises one or more channels formed into a distal face of the lower flange.

12. The surgical tool of claim 11, wherein the one or more channels extend radially outward from the lumen, the one or more channels being in fluid communication with the lumen.

13. A removeable tip for a surgical suction tube, comprising:
a gasket body having a proximal end and a distal end, with a lumen extending therebetween,
at least a portion of the gasket body comprising a flexible material,
the lumen including a proximal lumen end sized and configured to receive a distal end of the surgical suction tube,
the gasket body including a flange extending laterally outward from the distal end of the gasket body, and
a permeable patty or sponge attached to the flange, wherein at least a portion of the patty or sponge extends across the lumen at the distal end of the gasket body.

14. The removeable tip of claim 12, wherein the permeable patty or sponge is attached to a distal face of the flange.

15. The removeable tip of claim 12, wherein the flange includes a plurality of openings extending therethrough, and the permeable patty or sponge is attached to the flange by one or more sutures extending through the plurality of openings.

16. The surgical tool of claim 12, wherein the lumen is sized smaller in diameter than an outer diameter of the surgical suction tube, wherein the flexible material of the gasket body is capable of expanding to accommodate a distal end of the surgical suction tube.

17. The removeable tip of claim 14, wherein at least a portion of the permeable patty or sponge distally laterally beyond the distal face of the flange.

18. A surgical suction tube kit having a plurality of removable and replaceable tip elements, comprising:
an elongated hollow cylindrical tube connected to a vacuum source,
a plurality of removeable tips for placement on a distal end of the elongated hollow cylindrical tube, each of the plurality of removeable tips comprising an flexible gasket body having a proximal end, a distal end and a lumen extending therethrough, the lumen sized and configured with a smaller diameter than an outer diameter of the distal end of the elongated hollow cylindrical tube, wherein the flexible gasket body is capable of stretching the lumen to a larger diameter to accommodate the outer diameter of the distal end of the elongated hollow cylindrical tube therein,
each of the plurality of removeable tips including a permeable patty or sponge attached to a distal end of the gasket body, wherein when one of the removeable tips is attached over the distal end of the elongated hollow cylindrical tube, a vacuum from the vacuum source passes through the permeable patty or sponge and draws fluid from a body cavity through the permeable patty or sponge and into the elongated hollow cylindrical tube.

19. The surgical suction tool kit of claim 18, wherein the plurality of removeable tips comprises a plurality of different sized flexible gasket bodies.

20. The surgical suction tool kit of claim 18, wherein the plurality of removeable tips comprises a plurality of different sized permeable patties or sponges.
